(19) United States
(12) Patent Application Publication
(10) Pub. No.: US 2013/0299549 A1
(43) Pub. Date: Nov. 14, 2013

(54) PLICATION TAGGING DEVICE AND METHOD

(71) Applicant: Boston Scientific Scimed, Inc., Maple Grove, MN (US)

(72) Inventors: Samuel Crews, Woodside, CA (US); Bretton Swope, San Francisco, CA (US); Justen England, San Francisco, CA (US); David Cole, San Mateo, CA (US); Carlos Castro, San Jose, CA (US)

(73) Assignee: Boston Scientific Scimed, Inc., Maple Grove, MN (US)

(21) Appl. No.: 13/869,932

(22) Filed: Apr. 24, 2013

Related U.S. Application Data
(63) Continuation of application No. 12/434,226, filed on May 1, 2009, now abandoned.

(51) Int. Cl. A61B 17/068

(52) U.S. Cl. CPC A61B 17/068
USPC 227/175.1

(57) ABSTRACT

An improvement in a stapling device and method designed to capture a tissue fold between first and second members in the device to staple and cut the fold to form a stapled tissue plication with a hole therein. The improvement includes an engagement assembly in the first member movable from a retracted position in the first member to an extended position in the second member, and releasably attached to second member, an anchor assembly that includes the anchor, wherein (i) movement of the engagement assembly from the retracted to extended position, through a tissue fold captured in the device, is operable to engage the anchor assembly, and (ii) movement of the engagement assembly, with the anchor assembly engaged therewith, to its retracted position, is effective to pull at least a portion of the assembly through the hole in the stapled tissue plication.

### CROSS-REFERENCE TO RELATED APPLICATION

The present application is a continuation of U.S. patent application Ser. No. 12/434,226 filed on May 1, 2009.

### FIELD OF THE INVENTION

The present invention relates to the field of devices and methods for use in preparing a tissue region, such as an internal region of the stomach, for anchoring an implant, such as a food-restrictive pouch, at the tissue region.

### BACKGROUND OF THE INVENTION

Non-invasive surgery to attach a medical implant within the body, e.g., within the interior of a hollow organ such as the stomach, has become an important surgical option. For example, bariatric surgery to limit food intake into the stomach, in the treatment of obesity, can now be done transorally, rather than having to cut into or penetrate the peritoneal cavity. In a transoral procedure, an access tube is placed in the patient's esophagus, as a guide for one more or more endoscopic tools used in attaching an implant to, and/or reconfiguring, the stomach.

An anatomical view of a human stomach S and associated features is shown in FIG. 1. The esophagus E delivers food from the mouth to the proximal portion of the stomach S. The z-line or gastro-esophageal junction Z is the irregularly-shaped border between the thin tissue of the esophagus and the thicker tissue of the stomach wall. The gastro-esophageal junction region G is the region encompassing the distal portion of the esophagus E, the z-line, and the proximal portion of the stomach S.

Stomach S includes a fundus F at its proximal end and an antrum A at its distal end. Antrum A feeds into the pylorus P which attaches to the duodenum D, the proximal region of the small intestine. Within the pylorus P is a sphincter that prevents backflow of food from the duodenum D into the stomach. The middle region of the small intestine, positioned distally of the duodenum D, is the jejunum J.

Several co-owned applications, including U.S. Publication No. US 2007/0276432 having a priority date of Oct. 8, 2004 and U.S. Publication No. US 2008/0065122, filed May 23, 2006 describe methods according to which medical implants are coupled to tissue structures, such as plications or folds, formed within the stomach. Examples of methods and devices for forming such tissue structures are described in U.S. Publication No. US 2007/0219571 (entitled ENDOSCOPIC PLICATION DEVICES AND METHOD), filed Oct. 3, 2006, U.S. application Ser. No. 11/900,757 (entitled ENDOSCOPIC PLICATION DEVICE AND METHOD), filed Sep. 13, 2007, and U.S. application Ser. No. 12/050,169 (entitled ENDOSCOPIC STAPLING DEVICES AND METHODS), filed Mar. 18, 2008. Each of the referenced publications and applications is incorporated herein by reference.

As disclosed in these prior applications, more robust and long lasting coupling between the implant and the surrounding stomach wall tissue is achieved when the plications/folds are formed by retaining regions of serosal tissue (i.e., the tissue on the exterior surface of the stomach) in contact with one another. Over time, adhesions form between the opposed serosal layers. These adhesions help to create strong bonds that can facilitate retention of the plication fold over extended durations, despite the forces imparted on them by stomach movement and implanted devices

Several of the disclosed methods for forming tissue plications include a step in which a hole or cut is formed in the plication, using the plication forming device or a separate tissue-cutting device. Typically, the device also fastens the fold with an array of staples that are formed in the tissue about the hole. An example of this type of stapled tissue plication is shown in FIG. 2A, which is a cross-section taken along line 2B-2B in FIG. 1. Stapling devices and methods for forming such stapled tissue folds of this type are described in co-owned U.S. application Ser. No. 11/542,457, entitled ENDOSCOPIC PLICATION DEVICES AND METHODS, filed Oct. 3, 2006, and published Sep. 20, 2007 as US 2007-0219571, and co-owned U.S. application Ser. No. 12/050,169, entitled ENDOSCOPIC STAPLING DEVICES AND METHODS, filed Mar. 18, 2008, both and incorporated herein by reference. In a typical procedure that uses the stapled plications for implant attachment, a plurality of stapled tissue plications, each with an anchor-receiving hole, are formed in a tissue, such as illustrated in FIG. 2B. In this figure, five such plications are formed in the interior of the stomach, for attaching a food-restricting pouch near a patient's esophagus, to limiting food intake by the patient. After formation of the plications, an implant-retaining anchor is placed in each hole, and the implant, e.g., stomach pouch, is attached to the plications by introducing, for each plication, an anchor that extends through the hole and through an anchor-receiving opening in the implant. By way of illustration, for placement of a stomach pouch attached to five plications formed within the stomach (FIG. 2B), the implant operation will require ten separate steps in which an endoscopic device is placed in and then removed from the stomach transorally: five for forming each of the stomach placations, and five for each anchor placement between a placation and pouch anchor-receiving opening in the pouch.

Given the surgical time and inconvenience, and the patient discomfort, associated with each transoral-accessing step, it would be desirable to reduce the number of accessing steps needed for attaching an implant to a tissue placation. In the present invention, this is achieved by an improved device and method for forming a tissue plication that involves placing an anchor in the plication at the time the plication is formed.

### SUMMARY OF THE INVENTION

In one aspect, the invention includes an improvement in a stapling device designed to capture a tissue fold between first and second members in the device and to staple and cut the fold to form a stapled tissue plication with a hole therein. The improvement, for placing in the stapled tissue plication, an anchor effective to attach an implant device to the tissue fold, comprises an engagement assembly in the first member movable from a retracted position in the first member to an extended position in the second member, and releasably attached to second member, an anchor assembly that includes the anchor. Movement of the engagement assembly from its retracted to its extended position, through a tissue fold captured in the device between the first and second device members, is operable to engage the anchor assembly, and (ii) movement of the engagement assembly, with the anchor assembly engaged therewith, back toward its retracted position, is effective to pull a portion of the assembly through the hole in the stapled tissue plication formed by the device.

The first member in the device may include a housing and a drive piston movable within the housing from a retracted position to an extended position and a tissue cutter, the engagement assembly includes an engagement pin, the cutter and pin are attached to the drive piston for movement therewith, and movement of the drive piston from its retracted to its extended position is operable to (i) drive the pin through a tissue fold captured in the device, (ii) form a hole in the tissue fold, and (iii) engage the pin with the anchor assembly.

The first member may further include a staple holder having a first tissue-contact surface adapted to contact one face of a captured tissue fold and a staple pusher attached to the drive piston for movement therewith within the first-member housing, and movement of the drive piston from its retracted to its extended position is operable to (i) staple the tissue fold captured in the device, (ii) drive the pin through a fold, (iii) form a hole in the tissue fold, and (iv) engage the pin with the anchor assembly.

The second member in the device may include a housing, an anvil attached to the housing and having a second tissue-contact surface adapted to contact the other face of a captured tissue fold, and a cutter board mounted adjacent the anvil, against which the cutter is pressed when the cutter is moved to its second position, to cut a hole in a tissue fold captured between the tissue-contact surfaces of the staple holder and anvil.

The anchor assembly may include (i) a pin-engaging plug mounted releasably on the second-member housing adjacent the cutter board, (ii) an anchor and (iii) a tether that attaches the plug to the anchor. The cutter board may include a slot in which the tether is received, below the surface of the board. In this embodiment, movement of the engagement pin from its extended toward its retracted position, after engagement with the plug, is effective to (i) release the plug from the second member, (iii) pull the plug through a hole formed by the device in the staple tissue plication, and (iii) remove the tether from its slot in the cutter board.

The engagement pin and plug may be designed to allow the pin to penetrate a portion of the plug, as the pin is moved toward its extended position, but prevent disengagement of the pin with the plug when the pin is moved toward its retracted position.

The device may include a membrane connecting the two members, defining a tissue-capture chamber therebetween, and an opening in the membrane through which tissue can be drawn into the chamber, under the influence of a vacuum applied to the chamber, with the anchor in the anchor assembly held releasably on the device outside the chamber, and the tether connecting the plug to the anchor extending through the opening in the membrane. In this embodiment, movement of the engagement pin from its extended to retracted position, after engagement with the plug, is effective to (i) release the plug from the second member, (iii) pull the plug through a hole formed in the staple tissue plication, (iii) remove the tether from its slot in the cutter board, and (iv) release the anchor from the device, wherein the device can be manipulated to pull the anchor through the hole in the stapled tissue plication after the stapled tissue plication has been released from the device.

The anchor may include a front, compressible cap joined to a flexible back plate, where manipulating the device to pull the anchor through a hole in a staple tissue plication is to pull the cap through the hole, to engage one side of the fold, with the plate supported against the other side of the fold.

The improvement may further includes means for cutting the anchor-assembly tether, once the anchor is pulled through the hole in the stapled tissue plication. Alternatively, the tether may be designed to separate upon application of a predetermined pulling force applied to the tether.

The two members in the device may be relatively movable toward one another to capture a tissue fold therebetween, and relatively movable away from one another to release a stapled tissue plication from the device, prior to manipulating the device to pull the anchor through the hole formed in the stapled tissue plication.

In another general embodiment, the anchor assembly includes a front, compressible cap joined to a flexible back plate, the anchor is carried releasably within the second-member housing, movement of the engagement pin from its retracted to extended position is effective to engage the anchor, and movement of the engagement pin from its extended toward its retracted position is effective to (i) release the anchor from the second-member housing, and (ii) pull the cap through the hole in the stapled tissue placation formed by the device, to engage one side of the fold, with the plate supported against the other side of the fold.

The second-member housing may include a central cavity having an opening surrounded by the cutter board and anvil, with the anchor assembly being releasably contained within the cavity.

The engagement pin may includes means for releasing the anchor after the anchor's compressible cap has been pulled through the hole in the stapled tissue plication formed by the device.

Also disclosed is a method for forming a stapled tissue plication having placed therein, an anchor effective to attach an implant device to the tissue fold, by the steps of:

(a) capturing a tissue fold between two tissue-contact members,

(b) stapling the captured tissue so as to form a pattern of staples about an anchor zone,

(c) extending an engagement assembly through the anchor zone, before, during, or after said stapling step, to engage an anchor assembly,

(d) before, during or after said extending step, forming a hole in said anchor zone,

(e) retracting the engagement assembly and engaged anchor assembly to pull at least a portion of the anchor assembly through the hole at the tissue anchor zone, and

(f) releasing the stapled tissue from the tissue-contact members.

In one embodiment, the engagement assembly includes an engagement pin, the anchor assembly includes a pin-engaging plug, an anchor having a front, compressible cap and a back plate, and a tether connecting the plug to the plate, step (c) is effective to engage the pin with the plug, and step (e) is effective to pull the plug through the hole formed in the tissue in step (d). The method of this embodiment may further include, following step (f), manipulating the engagement pin to pull the anchor cap through the hole formed at the anchor zone in the stapled tissue plication, to engage one side of the fold, with the anchor plate supported against the other side of the fold, and cutting the tether to release the stapled tissue plication with anchor from the pin and engaged plug.

In another embodiment, the engagement assembly includes an engagement pin, the anchor assembly includes a front, compressible cap and a back plate, and a tether connecting the plug to the plate, step (c) is effective to engage the pin with the anchor, and step (e) is effective to pull the plug through the hole formed in the tissue in step (d), to engage one side of the fold, with the anchor plate supported against the other side of the fold, and which further includes disengaging the engagement pin from the anchor prior to releasing step (f).

These and other objects and features of the invention will become more fully apparent when the following detailed description of the invention is read in conjunction with the accompanying drawings.

### BRIEF DESCRIPTION OF THE DRAWINGS

FIG. 1 is a schematic illustration of a human stomach and a portion of the small intestine.

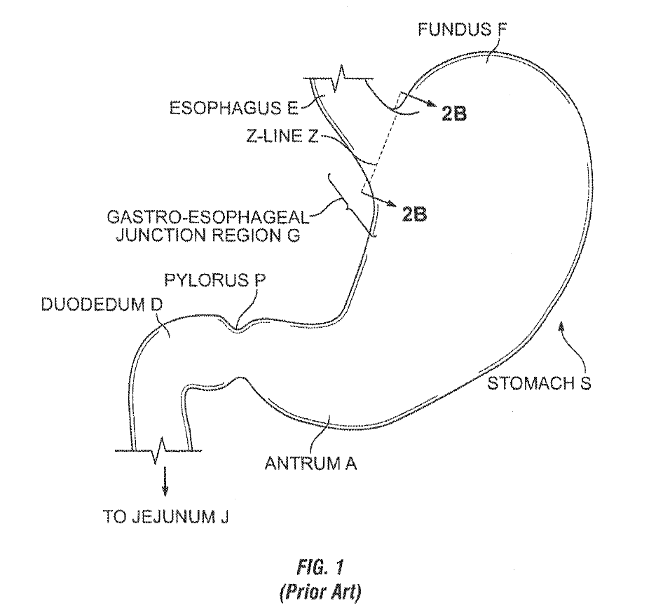

FIG. 2A is a partial section of a stomach wall showing a stomach wall plication having an opening formed in it.

FIG. 2B is a cross-section view taken along the plane designated 2B-2B in FIG. I, and illustrating five plications formed in a gastro-esophageal junction region of the stomach.

FIG. 3 illustrates an endoscopic stapling system or instrument constructed in accordance with an embodiment of the invention.

FIGS. 4A-4B are perspective views showing the stapler head or device of the stapling system of FIG. 2 in different positions.

FIG. 5 is a perspective view of the stapler head or device, with the membrane removed, showing first and second members in the device.

FIGS. 6A and 6B are perspective views of the proximal-member and distal-member housings in stapler device of FIG. 5.

FIG. 7 is an exploded perspective view showing elements advanceable within the staple housing during compression and stapling operations.

FIG. 8 is a perspective view of the staple housing similar to FIG. 6A, but showing some of the elements of FIG. 7 within the housing.

FIGS. 9A-9D are a series of schematic representations of the hydraulic chamber and pistons, illustrating operation of an exemplary hydraulic system during tissue compression, stapling, cutting and anchor engagement.

FIG. 10 is a perspective view of the anvil-member housing of the stapler device of FIG. 5.

FIG. 11 is a perspective view of the anvil support separated from the anvil-member housing.

FIGS. 12A and 12B are perspective views of the portion of portions of the anvil member and its anchor-assembly plug;

FIGS. 13A-13C illustrate the interaction of the engagement pin and anchor-assembly plug during operation of the stapler device, where FIG. 13C shows the anchor, anchor and connecting tether in an anchor assembly;

FIGS. 14 and 15 show portions of a stapler device in accordance with alternative embodiment of the invention, where the engagement assembly includes a pin (FIG. 14) or grasper (FIG. 15) and the anchor assembly includes an anchor received in a central cavity in the anvil member of the device;

FIG. 16A-16F illustrate steps in forming a stapled tissue placation with an attached anchor, in accordance with one embodiment of the invention;

FIGS. 17 and 18 illustrate a portion of a stapled tissue placation before (17), and after (18) anchor placement;

FIG. 19 illustrates a food-restrictive implant attached to a plurality of anchors.

### DETAILED DESCRIPTION

The present application describes a device, system and method for forming a tissue placation fold having placed therein, an anchor capable of attaching an implant to the fold.

In the disclosed embodiments, tissue is drawn inwardly into a vacuum chamber, although tissue may be drawn inwardly using other components (e.g. graspers) that do not involve the use of a vacuum. When a portion the interior stomach wall is drawn inwardly, sections of serosal tissue on the exterior of the stomach are positioned facing one another. The disclosed device allows the opposed sections of tissue to be moved into contact with one another, and delivers fasteners that will hold the tissue sections together until at least such time as serosal bonds form between them. As part of the same operation, an anchor capable of attaching an implant to the plication is placed in the plication, typically through a hole formed in the placation about an array of staples used in fastening the plication. That is, the disclosed device may include an optional feature that forms a hole or cut in a plication Each of these steps—reconfiguring tissue into a tissue fold or plication, stapling the fold, cutting a hole in the tissue, before, during or after the stapling step, and placing an anchor in the hole of the stapled tissue plication—may be performed wholly from the inside of the stomach, and may be all carried by a single device, without having to remove the device from the stomach. After one or more plications is formed, a medical device, such as a food-restricting pouch or restrictor, can be attached to the tissue through one or more of the placed anchors.

In the description of the embodiments given below, the device of the invention is described as being a stapler, and exemplary methods are given with respect to the formation of stapled plications in stomach tissue. It should be understood, however, that the embodiments described herein include features having equal applicability for applying other types of fasteners. More specifically, the term “staple” is used herein to designate any type of fastener that (i) can be pushed through tissue, and (ii) has one or more leg members that when forced against an anvil are crimped to secure the fastener to the tissue and hold tissue fastened tissue fold together, or (iii) a fastener having a rivet arrangement in which tissue is fastened by interlocking members on either side of the tissue. Similarly, the term “stapled” plication refers to a tissue fold fastened by any such fastener. The disclosed embodiments and methods will also find use in parts of the body other than the stomach. Additionally, although the disclosed embodiment features circular stapling and cutting of a concentric hole, and placement of an anchor is a circular hole, modifications are conceivable in which linear stapling can be accomplished, as well as circular or linear stapling without cutting.

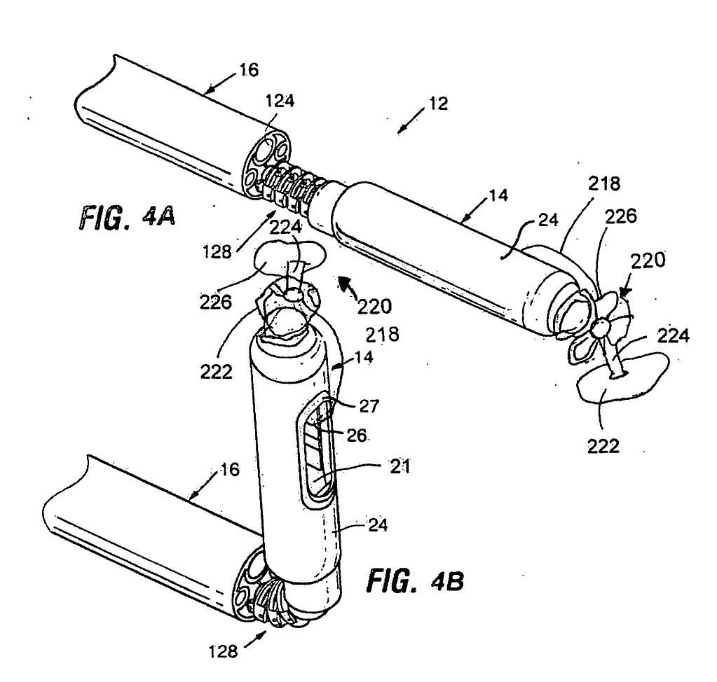

FIG. 3 illustrates one embodiment of a system or instrument 10 for tissue stapling and anchor placement that is suitable for endoscopic use, as well as surgical or laparoscopic use if desired. Generally speaking, system 10 includes a stapler instrument 12 having a stapler head or device 14 positioned on a distal portion of a shaft 16. Although device 14 is generally referred to herein as a stapler or stapler device, it will be understood that the device also includes functions for anchor placement, and preferably, as a cutter for cutting a hole in a stapled tissue plication through which the anchor is attached to the plication. A handle 18 on the shaft 16 controls articulation of the stapler head 14 and actuation of the device for tissue acquisition, tissue compression, stapling and cutter functions, and manipulation of the device for anchor placement. Vacuum and fluid sources 20, 31 in the system are fluidly coupled to the handle 18 for use in tissue acquisition, compression, stapling, cutting and anchor manipulation, as discussed below. The vacuum source 20 may be the “house vacuum” accessible through a coupling on the wall of the operating room, or an auxiliary suction pump. The stapler may include a switch 19 allowing the user to control airflow between the vacuum source and stapler.

The stapler device also serves to capture a tissue fold for stapling, and is thus also referred to herein as a tissue capture device for immobilizing a tissue fold, e.g., for fastening the sides of the fold. The tissue capture device may operate independently for capturing tissue, e.g., absent a separate stapling mechanism, or may be combined with the stapling elements, as illustrated. In one method described below, the tissue capture device functions to capture a tissue fold, and without a separate stapling and cutting step, fastens the tissue by applying a rivet type anchor to opposite sides of the plication, where the two parts of the anchor also function as anchors for attaching an implant to the fastened plication.

The fluid source 31 may be a single source of drive fluid (e.g. water, saline, oil, gas) or multiple sources, but in each case the fluid source preferably includes two actuators separately used to control flow into each of two hydraulic lines (one for tissue compression and one for stapling, cutting, and anchor manipulation). An endoscope 22 in the system is insertable through a lumen in the shaft 16 permits visualization of the plication procedure. The system may optionally include an overtube, such an endoscopic guide tube 23, having a lumen for receiving the stapler 12 and endoscope.

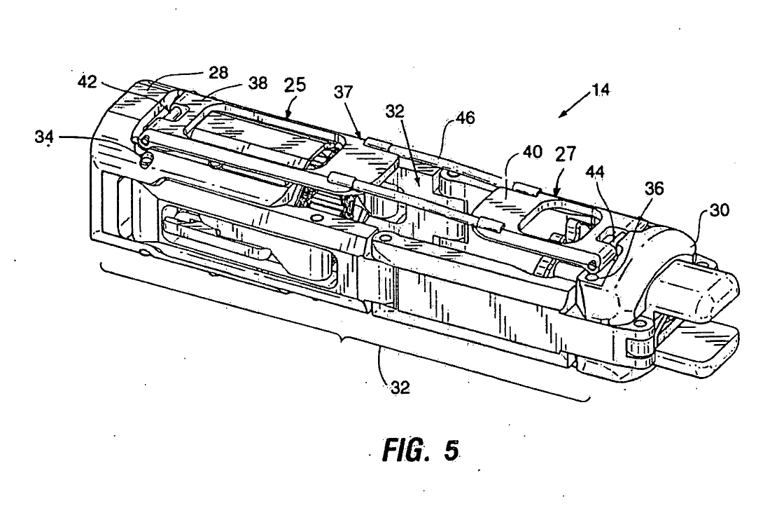

Referring to FIG. 4A, a covering or membrane 24 in device 14 encloses the stapler, cutter, and anchor placement mechanism to form a vacuum chamber 21 within the device. The side exposed to the tissue to be plicated remains uncovered by the membrane 24 to allow tissue to be drawn into the chamber during use. For example, the membrane 24 may include a side opening 26 as shown in FIG. 4B. Membrane 24 is preferably formed of silicone, elastomeric material, or any other inelastic or elastic flexible or deformable biocompatible material capable of forming a vacuum chamber 21 that will expand in volume to accommodate tissue drawn into chamber 21. Also shown in FIGS. 4A and 4B is an articulating section 128 connecting the instrument shaft to the staple head, allowing the head to be moved angularly with respect to the shaft, as described in the above-cited. U.S. application Ser. No. 12/050,169.

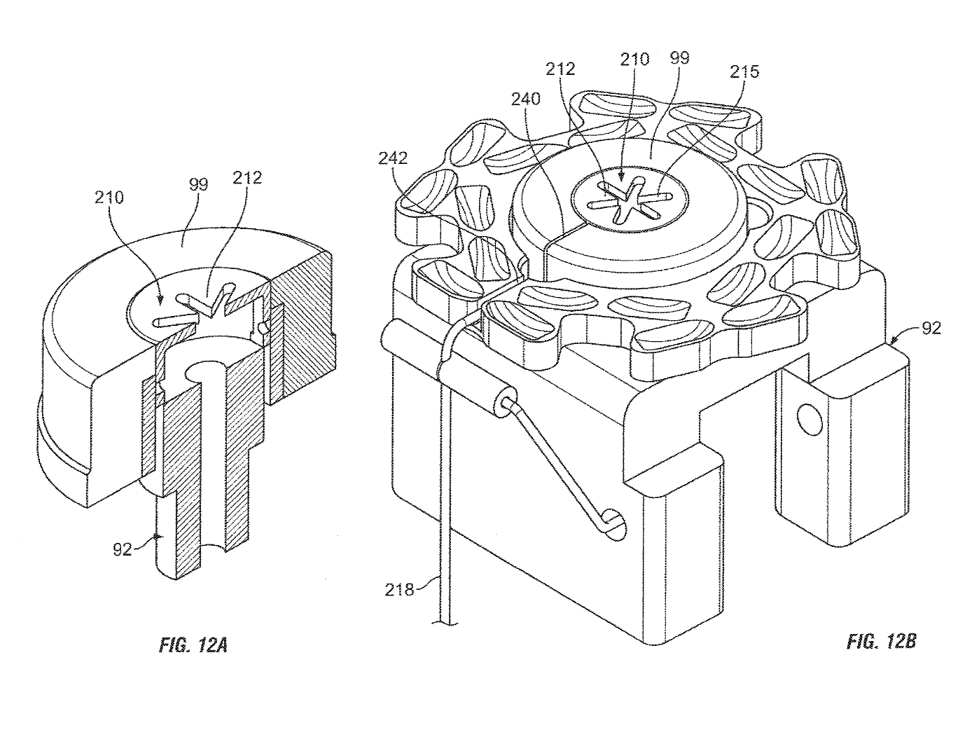

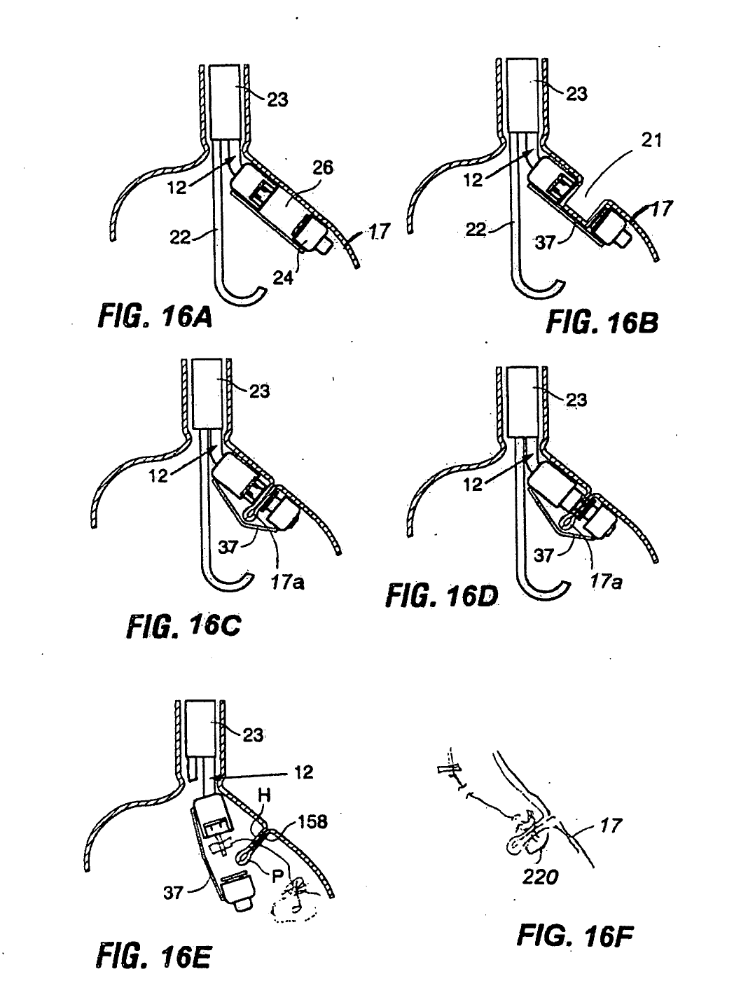

At least a portion of the membrane is partially transparent, allowing the user to see through the membrane well enough to confirm (via endoscopic observation) that an appropriate volume of tissue has been acquired into the stapler head prior to staple application. The opening 26 may be surrounded by a reinforced section 27 formed of material that will strengthen the area around the opening 26. Reinforced section 27 may be formed of a thicker section of the membrane material, and/or a higher durometer material. Alternatively, reinforcing ribs or other structures or elements may be formed into or onto the membrane material, or embedded in the membrane material. Also shown in the two figures is an anchor 220 releasably attached to the distal end of the stapler device, for example by an elastomeric loop in the anchor that encircles the end of the device. The anchor, which includes a front compressible cap 222, a stem 224, and a back flexible plate 226, is attached to plug carried within the chamber (described below with respect to FIGS. 11-13) by a tether 218 extending through opening 26 in the membrane. As will be detailed below with respect to FIGS. 16E and 16F, movement of the plug within the chamber during a plug-capture operation functions to pull the anchor from the device, allowing the device to be manipulated, once the stapled plication is released from the device chamber, to pull the anchor through a hole in the plication, after which the tether is severed.

### Stapler Head or Device

The stapler device of the present invention is designed to capture a tissue fold between first and second members in the device and to staple and cut the fold to form a stapled tissue plication with a hole therein. One such device is disclosed in the above-cited U.S. application Ser. No. 12/050,16911/542,457, published Sep. 20, 2007 as US 2007-0219571. This device includes a staple and an anvil housing that are relatively movable toward and away from one another to capture a tissue fold between staple-holder and anvil surfaces in the two members, and a separate drive piston that operates to apply an annular array of staples to the captured tissue fold, and optionally, operates to form a hole within the annular stapled zone in the tissue. Another suitable stapler device is the one detailed below, which includes, in addition to the features just described, the additional features that the staple holder and anvil move relative to their associated housings during a tissue-capture operation, to reduce the total distance the two housing have to move toward one another during a tissue-capture operation. In describing the construction and operation of a stapler device according to the present invention, which additionally involves placement of an anchor in a captured tissue plication, it will be understood how the modifications to a tissue capture and stapling device described below, for purposes of anchor placement, could be applied to any stapling device designed to capture a tissue fold between first and second members in the device and to staple and cut the fold to form a stapled tissue plication with a hole therein.

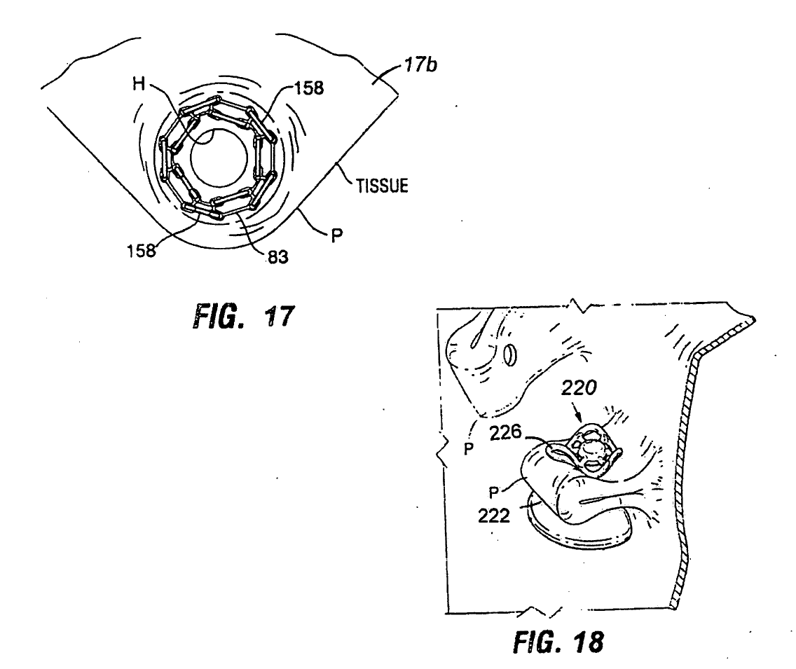

The stapler head 14 in the embodiment shown is designed to have a minimum profile during insertion to the plication site, and to then transform into a much larger profile device having a large internal volume. For example, in one embodiment the vacuum chamber might have an initial internal volume of 0.2 cubic inches, and an expanded volume of 0.6 cubic inches (i.e. the internal chamber volume after subtracting the volume occupied by the stapler head components positioned within the vacuum chamber). This large internal volume allows a large volume of tissue to be drawn into the vacuum chamber and stapled. In this way, the stapler head creates a large plication without requiring invasive techniques for insertion. The unique features of the stapler head allow in situ volumetric expansion of the stapler head using a minimum of motion and force input. In particular, as will be seen below with respect to FIG. 17, the plication can be sized such that the staples 158 applied to the tissue, shown at 17b, such that the two annular staple arrays seen in FIG. 17, are well spaced from the edges of the hole 83 formed in the stapled tissue, minimizing the risk of tissue tearing around the staples.

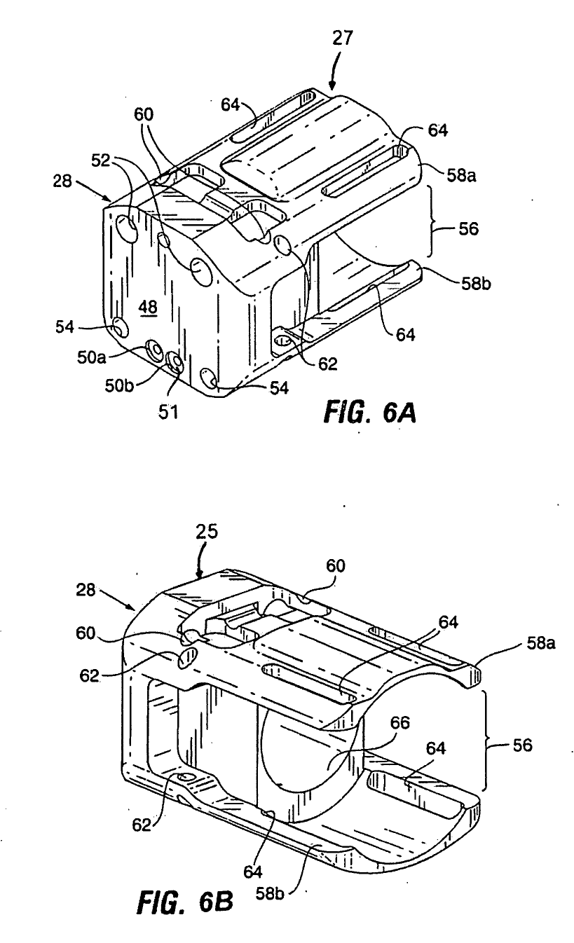

Features of the stapler head are shown in FIGS. 5-12. For clarity, the membrane is not shown in these figures. Referring to FIG. 5, stapler head 14 generally includes a first staple member 25 comprising a proximal staple housing 28, a second, anvil member 27 comprising a distal anvil housing 30, and at least one elongate member, but preferably a pair of hinged arm assemblies 32 that operatively connect the two housing as described below.

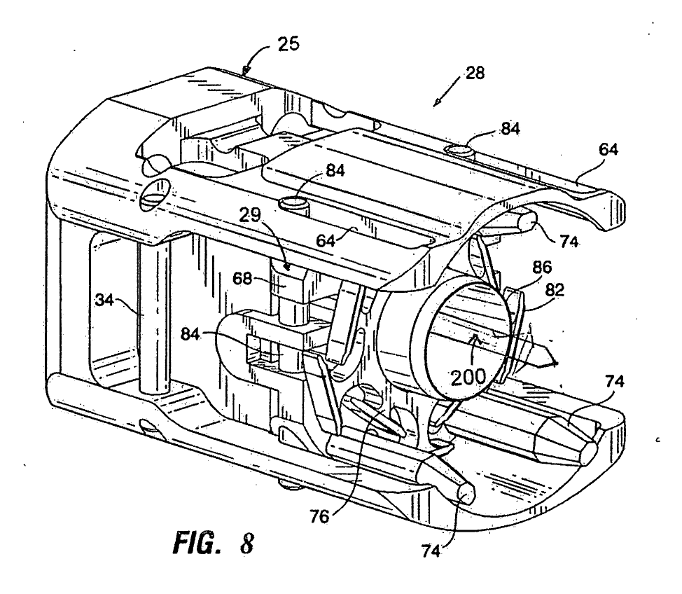

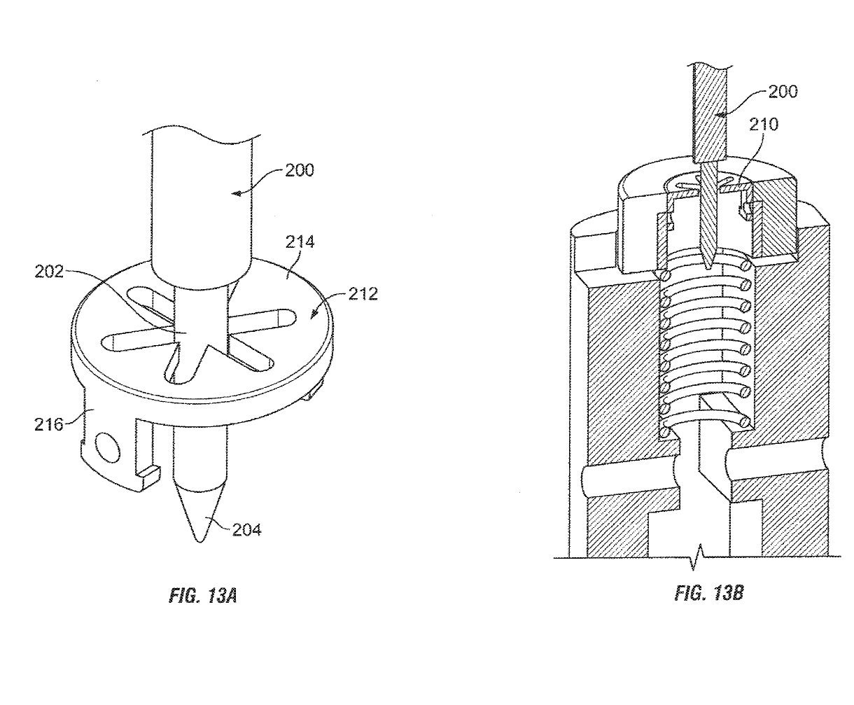

The staple housing and anvil housing are arranged to allow tissue to be compressed between contact surfaces on each of the staple housing and the anvil housing. In the disclosed embodiment, the contact surfaces are on a staple holding portion of the staple housing, i.e., the outer face of the staple holder, and an anvil on the anvil housing. Considering only the tissue-capture operation of the device, staple holder 78 (shown in FIG. 7) functions as a tissue-capture plate having a front, tissue-contacting surface 83, and anvil 96 (shown in FIGS. 11 and 12) functions as a second tissue-capture plate having a tissue contacting surface 103 that confronts surface 83, where these two surfaces serve to capture the tissue fold during operation of the device, as will be described more detail with respect to FIGS. 16A-16C.

With continued reference to FIG. 5, arm assemblies 32 extend between the staple housing 28 and anvil housing 30 on opposite sides of the stapler head 14. Proximal and distal pins 34, 36 pivotally couple each arm assembly 32 to the staple housing 28 and the anvil housing 30. An expansion member comprising a membrane raiser 37 also extends between the staple housing 28 and the anvil housing 30 (FIG. 5). Although the membrane 24 is not shown in FIG. 5, it should be understood that the membrane raiser 37 is positioned opposite the opening 26 (FIG. 4B) in the membrane. In the illustrated embodiment, membrane raiser 37 includes a link 38 pivotally mounted to the staple housing by a pin 42, a corresponding link 40 pivotally mounted to the anvil housing by pin 44, and spring wires 46 coupling the links 38, 40 to one another.

Staple Housing

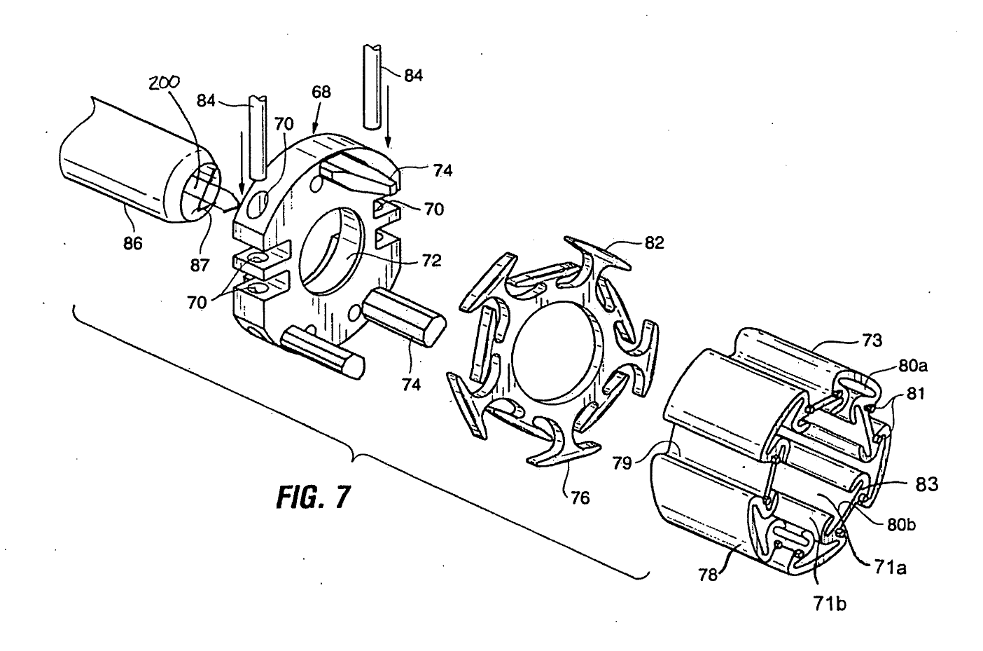

Turning to a more detailed discussion of the stapler head components, the staple housing 28 can be seen separated from other components in FIGS. 6A and 6B. As shown in FIG. 6A, proximal face 48 of the staple housing includes input ports 50a, 50b through which fluid is directed for hydraulic actuation of the tissue compression, stapling, and optional cutting operations of the stapler head. Seals 51 surround the ports 50a, 50b to minimize fluid leakage.

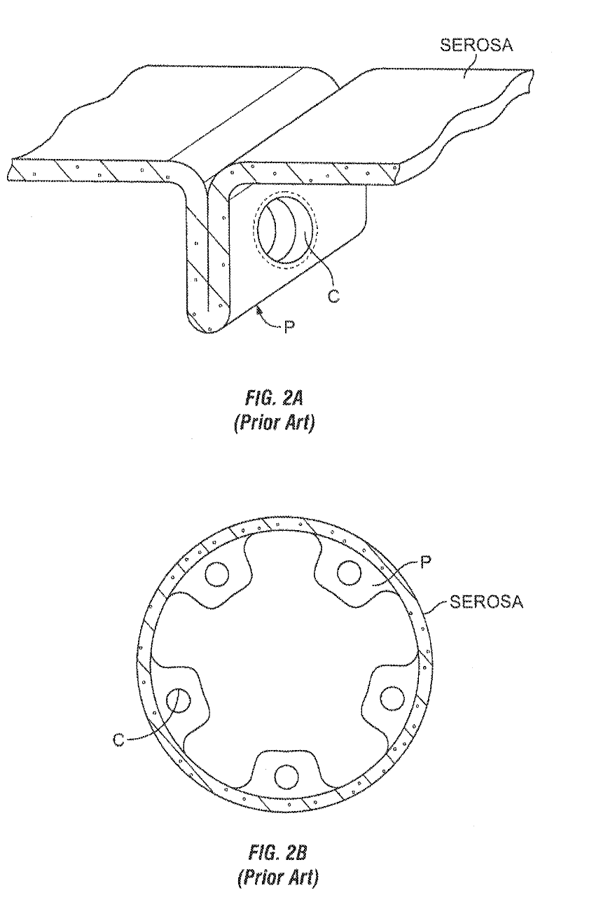

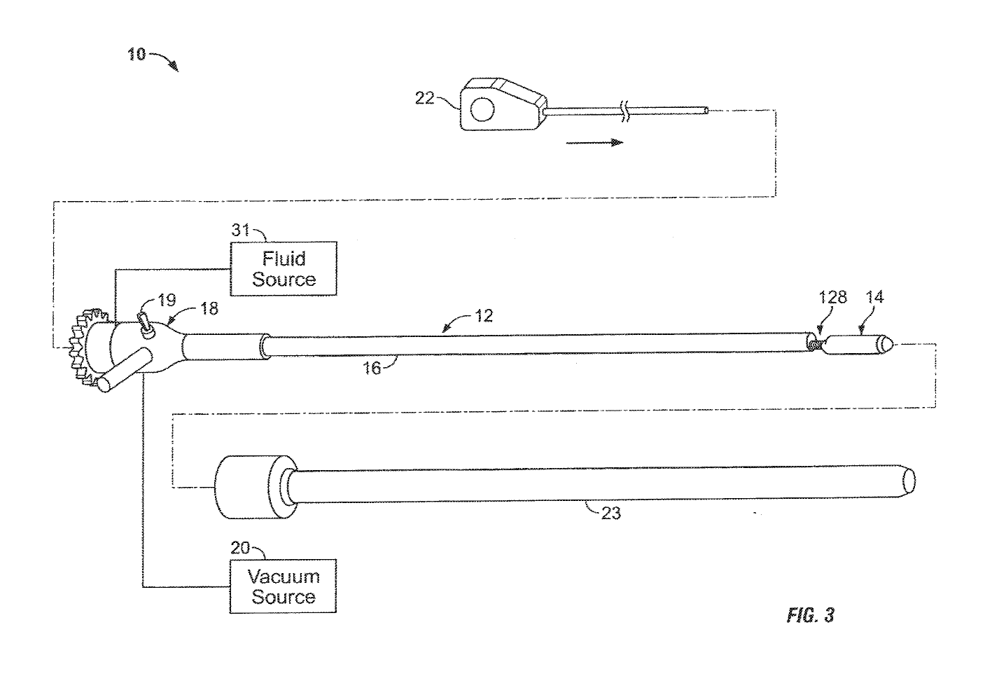

Vacuum ports 52 are fluidly coupled to a vacuum source 20 (FIG. 2) that is selectively activated to create negative pressure in the vacuum chamber for tissue acquisition. The vacuum ports 52 are connected to the vacuum source 20 by flexible tubing (not shown) in the stapler shaft 16 (FIG. 2). Mounting holes 54 are used to mount the stapler head 14 to the shaft 16 through the articulating section 128.

The staple housing 28 includes upper and lower sections 58a, 58b above and below open side sections 56. The upper section 58a includes a recess 60 within which the pivot pin 42 for link 38 (FIG. 5) is mounted. As best shown in FIG. 6B, bores 62 are positioned in the upper and lower sections 58a, 58b to receive pins 34 (FIG. 5) that serve as the proximal pivot points for arm assemblies 32. Guide slots 64 extend longitudinally through the upper and lower sections 58a, 58b.

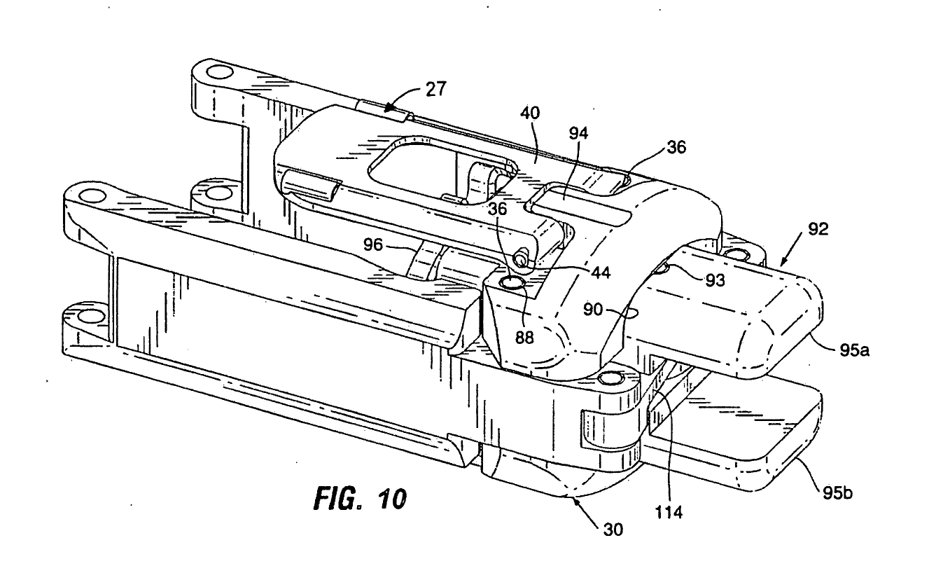

Referring to FIG. 6B, a hydraulic chamber 66 is disposed within the staple housing 28. Within the hydraulic chamber 66 (FIG. 6) is a dedicated hydraulic circuit for driving the tissue compression and stapling functions of the stapler, as described below with respect to FIGS. 9A-9D. Chamber 66 is fluidly coupled to the fluid input ports 50a, 50b (FIG. 5). As will be discussed in detail in connection with FIGS. 9A-9D, fluid driven into the hydraulic chamber 66 via input ports 50a, 50b sequentially advances a system of hydraulic pistons (not shown) that act on other components to compress the tissue, and that drive the staples and cutting element through the compressed tissue, and to engage an engagement assembly with an anchor assembly as the first step in the anchor placement operation.

FIG. 7 illustrates components of the stapler head that are driven by the hydraulic system for compression, stapling, and cutting. For clarity, these components are shown separated from the staple housing and from each other. In this discussion, the components that are driven by the hydraulic system will be described. The hydraulic system itself is described in a later section in connection with FIGS. 9A-9D.

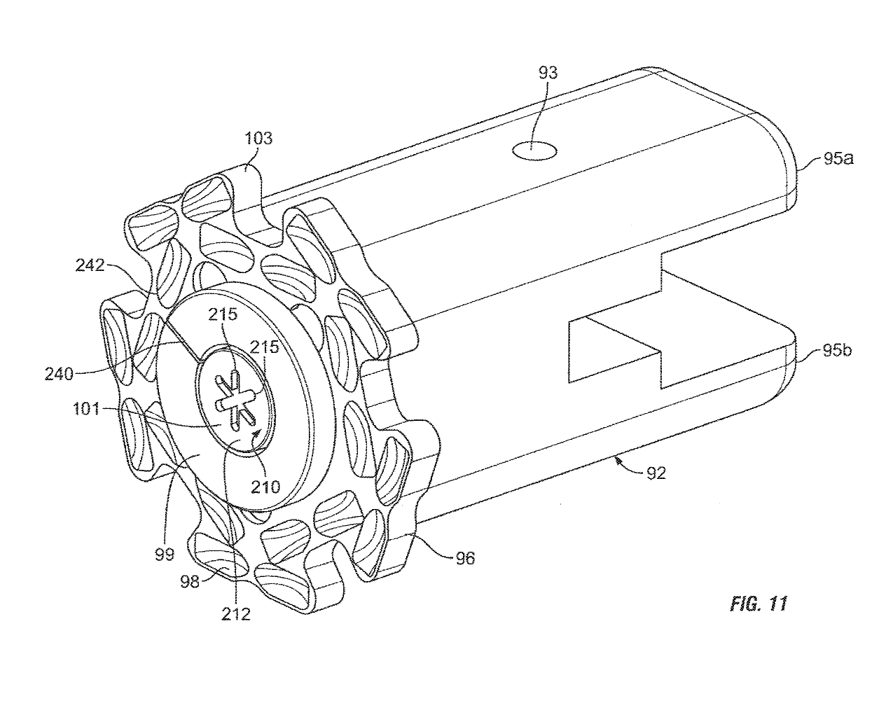

In particular, FIG. 7 illustrates a drive member which takes the form of a disk 68 in the staple housing. In the assembled housing, disk 68 is positioned such that it will be pushed distally by a hydraulic compression piston (piston 106 in FIGS. 9A-9D). The drive member is movable between a first, retracted position shown in FIG. 9A to a second, extended position shown in FIGS. 9C and 9D. Although the drive member illustrated here is driven by, but separate from piston 106, it will be appreciated that these two components can be formed of a single-piece member, i.e., as a single-piece drive member including both piston and disc. As will be seen below, the drive member is coupled to the arm assemblies 32, the anvil housing, and the staple housing so that advancing the drive member distally (toward its extended position) effects tissue compression by bringing the contact surfaces of the staple housing and anvil housing relatively towards one another. The combination of disk 68, its driving piston 106, and assembly arm 32 coupling the two housings is also referred to herein collectively as a drive assembly, indicated at 29 in FIG. 10. The drive assembly may further include drive links 114 in the anvil member, which are operatively linked to assemblies 32 as described below.

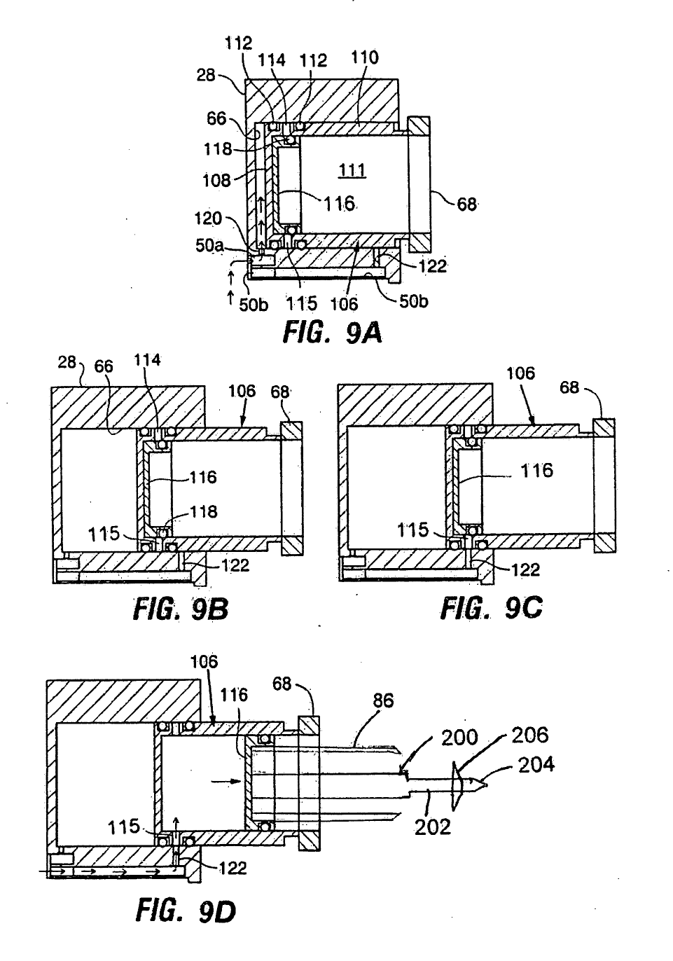

As seen best in FIG. 7, disk 68 includes mounting bores 70, a central opening 72, and alignment posts 74. Referring briefly to FIG. 8, in the assembled stapler head, disk 68 is coupled to the stapler housing 28, and its axial movement therein constrained, by pins 84 that extend through the housing's guide slots 64 and through mounting bores 70 in the disk 68.

A portion of the staple housing 28 contains, i.e., is loaded to contain, staples to be fired into the tissue. The staples are contained within a staple holder, such as staple cartridge 78, on the staple housing. The staple holder may have a number of different configurations. For example, it may be an integral portion of the staple housing, or a separate part mounted or attached to the staple housing, and/or it may be moveable relative to the body of the staple housing to effect tissue compression prior to stapling. In any of these examples, the staple holder may be a removeable/replaceable cartridge, and/or it may be refillable by inserting additional staples into it. In other embodiments, the staple holder may be neither replaceable nor refillable, i.e., in a dive intended for one-time use.

In the disclosed embodiment, the staple holder is a removeable staple cartridge 78 that can be replaced with another cartridge after staple filing. In this embodiment, the staple cartridge is moveable relative to the body of the staple housing to compress the tissue prior to staple firing.

With continued reference to FIG. 7, staple cartridge 78 is positionable within the staple housing, distal to the disk 68, such that distal advancement of the disk by the compression piston pushes the cartridge from a first, retracted position distally to a second, extended position to compress tissue disposed between the cartridge and anvil. Grooves 79 on the exterior of the cartridge slide over corresponding ones of the alignment posts 74 during insertion of the cartridge into the stapler head. FIG. 8 shows the alignment posts prior to loading of a cartridge into the staple housing. As shown, the alignment posts 74 may have tapered ends to facilitate loading of the cartridge over the posts. It will be appreciated that the alignment posts hold the cartridge against angular movement within housing 28 during stapler operation.

Again referring to FIG. 7, cartridge 78 includes a number of staple locations 80, each housing a staple, such as staples 158 seen in FIG. 33. The staple cartridge is equipped with bosses 81 to retain a staple line reinforcement device of the type shown disclosed in detail in above-cited, co-owned U.S. patent applications U.S. Ser. No. 11/542,457, published Sep. 20, 2007 as US 20070219571. To summarize briefly, this type of reinforcement device may be a ring or other element positionable against the distal face of the staple cartridge. When the ring is placed on the cartridge, openings in the ring align with prongs of some of the staples in the cartridge. When staples are driven from the cartridge, these prongs pass through associated ones of the openings 85 and capture the ring 83 against the adjacent body tissue.

In the embodiment shown, a cutter element 86 extends through the central opening 72 (FIG. 7) of the disk 68. The cutter element is shown as a tubular punch having a sharpened wall and a lumen 87, but may be provided in alternative forms. A staple pusher 76 is mounted to the cutter element, distally of the disk as can be seen in the assembled view of FIG. 10. Staple pusher 76 includes pusher elements 82 proportioned to slide into the cartridge's staple locations 80 as the staple pusher 76 is advanced into the staple cartridge 78, thus driving the staples from the cartridge. A hydraulically-driven staple piston (shown at 116 in FIGS. 9A-9D) in the hydraulic chamber 66 (carried within a hydraulic chamber formed by piston 106) is coupled to the cutter element 86 such that advancement of the stapler piston advances the staple pusher 76 and cutter element 86 in a distal direction, as shown in FIG. 9C.

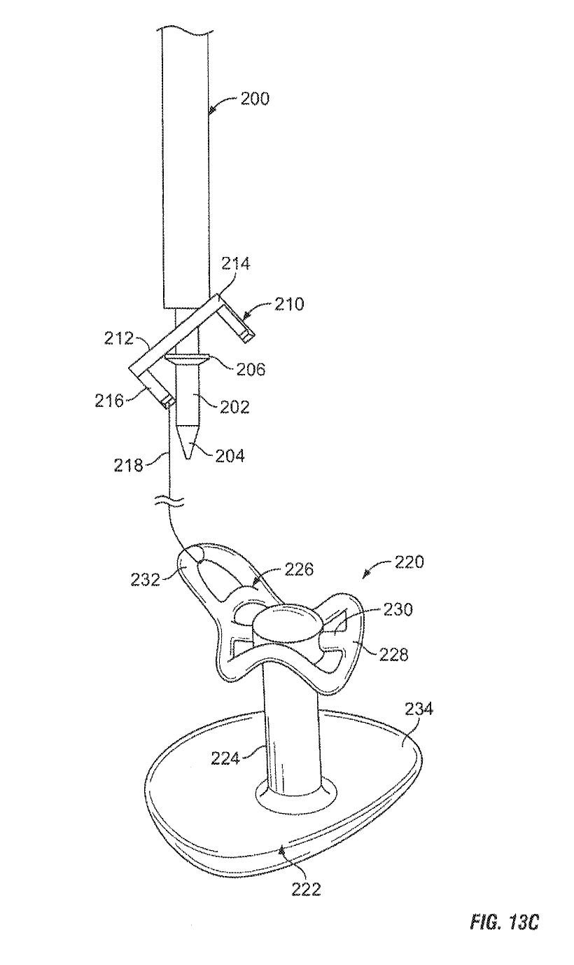

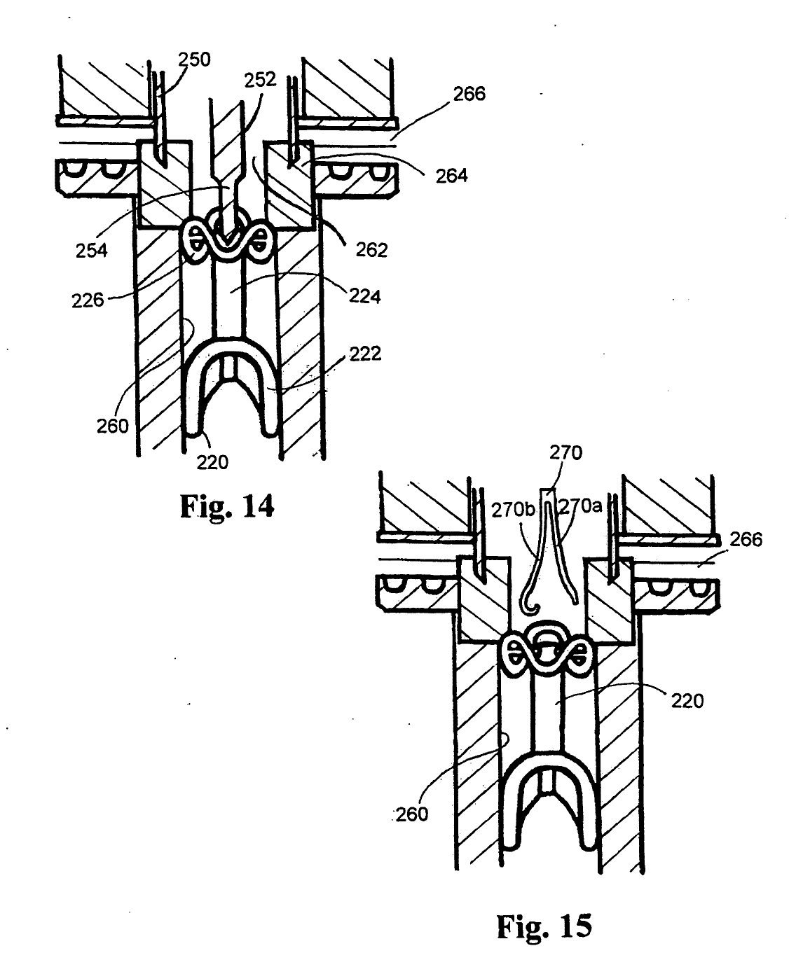

Also shown in FIG. 7 is an engagement pin 200 also carried on piston 116 (FIG. 9D) for movement therewith, between a retracted position and an extended position. As seen best in FIG. 9D, the pin is mounted on the piston for movement along the central axis of the piston. Pin 200, which is also referred to herein as an engagement assembly, includes a shaft 202 attached at its proximal end to the piston, and terminating at its distal end in a tapered end region 204. An annular ledge or projection 206 in the pin tapers outwardly in a distal-to-proximal direction, for engaging an anchor assembly, as will be described below with reference to FIGS. 13A-13C. Other embodiments of an engagement assembly in accordance with the invention will be described below with reference to FIGS. 14-16.

Fluid Drive System

The fluid drive system used to actuate tissue compression, stapling, cutting and anchor engagement may be configured in various ways. The following paragraphs describe one exemplary configuration for the fluid drive system, which in this embodiment is a hydraulic system. FIGS. 9A and 9B schematically show the fluid flow in the hydraulic chamber 66 of the staple housing 28 during both compression and stapling stages of actuation. Referring to FIG. 9A, compression piston 106 is disposed within hydraulic chamber 66. Disk 68 (also shown in FIGS. 7 and 8) is positioned in contact with or slightly distal to piston 106. Compression piston 106 is generally cup-shaped, having a rear wall 108 and a side wall 110 enclosing an interior 111. O-ring seals 112 are spaced-apart on a proximal portion of the side wall 110. Channels 115 are formed through the side wall 110, between the o-ring seals 112.

A second piston, referred to as the staple or drive piston 116, is positioned in the interior 111 of compression piston 106, against the rear wall 108. Although shown only in FIG. 9D, cutting element 86 (FIG. 7) and engagement pin 65 are carried axially on piston 116, for movement therewith. An o-ring seal 118 surrounds a portion of the staple piston 116 that is distal to the channels 115 in the compression piston.

A first fluid channel 120 extends from fluid port 50a in the stapler housing 28 to a proximal section of the hydraulic chamber 66. A second fluid channel 122 extends from fluid port 50b in the stapler housing to a more distal section of the hydraulic chamber 66. Fluid flow from port 50a and fluid channel 120 against the compression piston cylinder is shown in FIG. 9A. Fluid pressure within the hydraulic chamber 66 advances the compression piston 106, with the stapler piston 116 within in it, in a distal direction, from a first, retracted position, shown in FIG. 9A to a second, extended position shown in FIGS. 9C and 9D. FIG. 9B shows the compression piston 106 approaching the end of its travel, i.e., fully extended position. Once the compression piston reaches the end of its travel as shown in FIG. 9C, channel 115 in the compression piston 106 aligns with channel 122 in the housing, allowing fluid introduced through fluid port 50b to enter the interior of the compression piston 106 via channel 122. The fluid entering the interior of the compression piston drives the staple piston distally as shown in FIG. 9D, from a first, retracted position shown in FIGS. 9A-9C, to a second, extended position shown in FIG. 9D. In an alternative embodiment (not shown) a third, cutter piston is provided for separately driving the cutting element 86 and engagement assembly 200. In this embodiment, fluid introduced into a third drive fluid port causes advancement of the cutter piston from a first, retracted position to a second extended position (not shown). The pistons 106, 116 and the cutter piston and associated fluid paths may be arranged so that fluid cannot enter the interior of the stapler piston to advance the cutter piston 117 until compression piston 106 has traveled to the tissue-compression position and stapler piston 116 has in turn traveled to the stapling position.

The anvil housing (identified by numeral 30 in FIG. 5) in anvil member 27 will next be described with reference to FIG. 10. The anvil housing 30 includes mounting bores 88 for receiving pivot pins 36 at the distal end of the hinged arm assemblies 32. The upper section of the anvil housing 30 includes a section 94 through which the pivot pin 44 for link 40 (FIG. 5) is mounted.

A central bore 90 extends longitudinally through the anvil housing 30. An anvil support 92 is longitudinally slidable within the bore. Both the bore 90 and the anvil support 92 are preferably formed to have non-circular cross-sections with flat bearing surfaces to prevent rotation of the piston within the bore.

FIG. 11 shows the anvil support 92 separated from the anvil housing 30. The distal portion of the anvil support 92 is split into upper and lower plates 95a, b. Plate 95a has a bore 93 axially aligned with a similar bore in plate 95b. The proximal portion of the anvil support 92 carries the anvil 96. As seen in the figure, anvil 96 includes a plurality of indentations 98 positioned such when staples are driven from the staple cartridge, each staple leg engages one of the indentations, which causes the staple leg to fold or crimp. The surface of the anvil containing the indentations provides the anvil's tissue-contact surface 103. In the embodiment shown, the anvil is designed for a staple array having two annular rings of offset staples, five staples per ring. A central opening or cavity 97 extends through the anvil 96 and is contiguous with a lumen in the anvil support 92.

The anvil 96 and the staple cartridge 78 (FIG. 7) are the two parts of the stapler head which exert force on the tissue to be stapled. As shown in FIGS. 7 and 11, the preferred cartridge and anvil are designed to use a minimal amount of material surrounding the indentations 98 of the anvil 96 and the staple locations 80 of the cartridge 78, so that the amount of anvil/cartridge surface area contacting the tissue is as small as possible. When subjected to a constant force, a smaller footprint will damage less tissue than would a larger footprint, since a smaller area of tissue is squeezed between the anvil and cartridge. However, the tissue that does get squeezed experiences more pressure from the given force because the force is distributed over a smaller area. In other words, the minimized footprint creates more pressure on the tissue with less force. This is advantageous from a mechanical standpoint because the stapler head need not supply or withstand as much force as would be needed with a larger-footprint cartridge and anvil.

Referring to FIG. 7, in the illustrated embodiments, the staple cartridge 78 has an outer wall that tracks the contours of the staples housed within it, thus forming a number of pedals 73 surrounding the outer staple positions or slots 80a, with the grooves 79 disposed between the pedals, adjacent to the inner staple positions 80b. Rather than providing each staple position to be fully surrounded by cartridge material, the staple positions 80a, 80b preferably each include a back wall 71a and a retaining element attached to the wall and positioned to retain a staple between the retaining element and the back wall. In FIG. 7, the retaining element comprises a pair of wings 71b that curve inwardly from the back wall 71 to define a slot that is sufficiently bounded to retain a staple within the staple position, but that is preferably not bounded around its full circumference. The anvil has a similar pedal arrangement, as shown in FIG. 11.

With continued reference to FIG. 11, a cutter plate 99 is positioned on the anvil 96 such that the distally-advancing cutting element 86 will advance into contact with the plate 99 during tissue cutting. In the embodiment illustrated, plate 99 is positioned within the annular interior of the anvil. Plate 99, which will also be referred to as the “cutting board”, has a central hole 101 in it which relieves the pressure of the captured tissue and prevents hydraulic locking, a condition in which the punch and plate create a closed volume. If it is desired to move the cutting element 86 after contact is made, pressure will increase inside this closed volume and it will resist further motion. This may prevent or adversely affect tissue cutting.

The cutting board is preferably designed so as to provide a compressible stop against advancement of the cutting element 86. If cutting element 86 is stopped by the cutting board, the stapling piston will also be stopped and incomplete staple formation may result. Therefore, it is preferred that the cutting element 86 is allowed to penetrate or displace the cutting board during and after the tissue is cut; that is the cutter can advance slightly once initial contact with the board is made. To that end, the cutter board may be formed of a material that can be penetrated by the cutter blade, e.g., silicone, or may be supported on the anvil housing by a compressible member, such as an O-ring, that allows the board to move slightly in a distal direction after initial contact with the cutter blade.

Plication Tagging Components and Operation

Also shown in FIG. 11 is a plug 212 which forms part of an anchor assembly 210 shown in FIG. 13C. With reference to that figure, plug 212 in the assembly is formed of a rigid circular plate 214 having a pair of opposed legs 216, the plug being conveniently formed as a unitary object from a thin metal plate or semi-rigid molded plastic. The plug is attached, via a tether 218, to an anchor 220 in the assembly, as shown. The tether is preferably formed from a bioerodable suture material, such as polylactide, to allow it to biodegrade in the implant organ, e.g., stomach, over time.

With continued reference to FIG. 13C, the anchor 220 in the anchor assembly includes include a base 222, a stem 224, and a head or cap 226. The anchor is formed using materials that are durable within the stomach environment. In one embodiment, the head 32 is molded out of a higher durometer compliant material (such as 50 shore A durometer Silicone) while the stem 30 and base 28 are molded out of a softer compliant material (such as 5 shore A durometer Silicone). Since, as will be seen below, the engagement of the anchor with an implant can be seen as shear against the edges of the opening in the plication, the stem 224 is formed to have a relatively large diameter (2 mm-8 mm) to minimize stress and abrasion on the stomach wall tissue inside the opening. The edges of the anchor are molded with generous fillet radii to minimize abrasion of stomach wall tissue.

Cap 226 is formed of a ring 228 and a plurality of struts 230 coupling the ring to stem 224, and an secondary loop 232 extending from the ring and attached to tether 218. The anchor is elastically deformable to an elongated shape in response to application of tension to the cap, e.g., ring 228 or loop 232 (collectively referred to as the “rim”). This allows the anchor to be drawn into a streamlined shape so that it can be drawn through the opening in the plication and also through an opening in an implant. When the anchor is pulled from the rim, its shape lengthens and slims down to fit through a much smaller hole. For example in one embodiment, in its natural state the anchor has an outer head diameter of approximately 0.600 inch (15 mm), but in its streamlined orientation it can fit through a plication opening of 0.200 inch (5 mm). However, once implanted, the anchor's shape resists pull-out force to a higher degree since the rim is not being pulled and lengthened directly. Also in this embodiment, the base is designed so it will not pull through the hole and may have an outer diameter of approximately 1 inch (25.4 mm). Also as seen in the figure, the cap is shaped to have an undulating profile to enhance its visibility and accessibility when the anchor is positioned in a plication opening. The undulation of the head forces several of the elements of the head away from the plication wall to make them more visible and also to allow a grasping tool to latch onto one of those elements without also grabbing adjacent tissue.

With continued reference to FIG. 13C and also to FIG. 18B, base 222 of the anchor 222 is preferably formed to have an asymmetrical shape. In the illustrated example, one edge 234 of the base is formed to have a flatter curvature than that of the other edge of the base. When implanted, the anchor self-orients to position the flatter edge against the adjacent stomach wall as seen in FIG. 18B. Since the loop 232 of the cap extends in a direction opposite to the side of the anchor on which the flatter edge 234 is positioned, this self-alignment causes the loop 232 to extend towards the center of the stomach as shown in FIG. 18B. This makes it easier to find segments of the anchor head amongst the folds of the stomach which can envelope other segments.

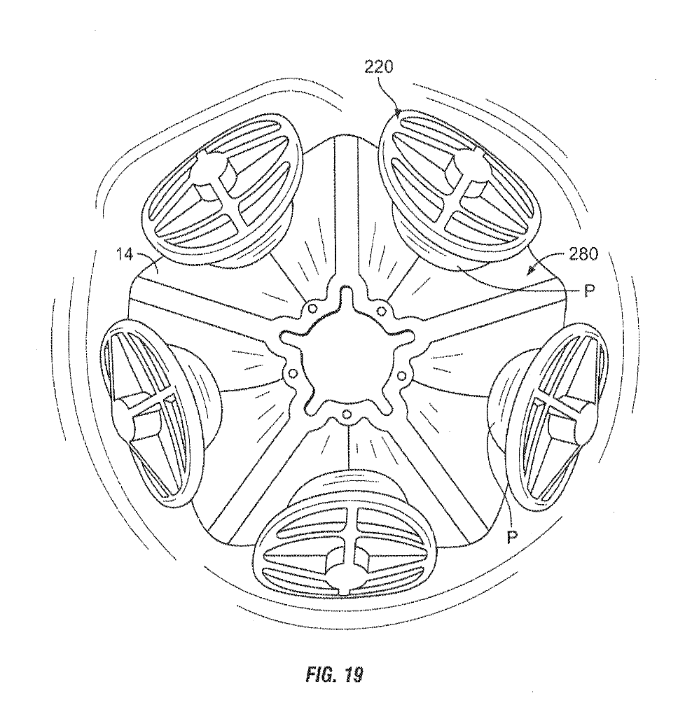

Base 222 of the anchor preferably has a relatively large surface area (e.g., approximately 1 square inch) so as to distribute the stress of holding the restrictive implant in place over a large percentage of the surface area of the tissue plication. Reinforcing ribs may be positioned on the underside of the base, radiating from the stem to the edges of the base, to facilitate distribution of stress while minimizing the overall weight of the base, as seen in FIG. 19.

With reference again to FIGS. 11 and 12, plug 212 is releasably carried on the anvil member within the annular hole in cutting board 99, that is in coaxial arrangement with the cutting board and anvil. Plate 214 of the plug has a star-shaped cutout pattern, indicated at 215, that allows engagement pin 200, and in particular, the ledge 206 on the pin, to penetrate the plate as the fingers of the star-shaped pattern on the plate are pushed down to accommodate the ledge in a distal direction, as the pin is moved with the cutter from its retracted to its extended position, seen in FIGS. 13A and 13B. Tether 218 which connects the plug to the anchor in the assembly, is disposed below the surfaces of the cutting board and anvil in slots 240, 242 in the board and anvil respectively, to prevent severing of the tether during the tissue stapling and cutting operations. Once the plate has been penetrated by the pin, the pin is captured by the plug; movement of the pin back toward its retracted position is effective to release the plug from first from the anvil member and pull the pin and attached plug through the hole formed in the stapled tissue plication. It is noted that the engagement pin and plate in the anchor-assembly plug also function to a maintain the two device members in axial alignment, during the stapling, cutting and engagement operations of the device.

Once the plate has been pulled away from its original position on the anvil member, tension on the tether causes the plate to assume a tilted position tilt on the pin, acting to lock the plug plate 214 on the pin, as seen in FIG. 13C. At the same time, movement of the pin and captured plug toward a retracted position pulls the tether out of the slots in the cutting board and anvil, and pulls the anchor off the distal end of the staple device, so that one end of the tether is attached to the retracted pin, and the other end of the tether is attached to the free-floating anchor. Now, when the stappled tissue plication is released from the device, the device can be manipulated with respect to the plication to pull the cap of the anchor through the plication hole, to place the anchor in the hole. The process is completed by severing the tether.

Although not shown in the drawings, a variety of means for severing the tether, once anchor placement has occurred, are contemplated. For example, where the plug, or the tethered leg of the plug is formed of a heat-conductive metal, supplying a heating current to the region of the pin in contact with the tethered plug leg (FIG. 13C) engagement pin would be effective to cut the tether by heat. Alternately, the staple housing may include a blade or separate cutting element that could be used to cut the tether after placing the anchor in the plication, or a separate endoscopic tool in the device could have a cutting blade for cutting the tether.

FIG. 14 illustrates portions of a stapling device for tissue plication stapling and anchor placement constructed in accordance with alternative embodiments of the invention. In the figure, a staple pusher (not shown), a cutter 240, and an engagement assembly 242 are all attached to a drive piston (not shown) in a staple member of the device. The engagement assembly includes an engagement pin 244 that terminates at its free end in a hook-like extension 245. As with the embodiment described above, movement of the drive piston between its retracted and extended positions moves the cutter blade and distal end of the engagement pin from the staple assembly toward and into the anvil assembly, while also ejecting staples for the stapling operation.

The anchor assembly in this embodiment includes an anchor 220 like that described above, having a web-like cap 226 attached to a plate 222 through a stem 224. The anchor is releasably carried, in a compressed form, in a central cavity 250 in the anvil member of the device, that is, the cavity whose opening coincides with the central opening 252 in the cutting board, indicated at 254. In operation, and with a tissue plication 256 captured between the staple holder and anvil surfaces, as detailed above, the drive piston for the staple pusher, cutter and engagement assembly is moved from its retracted position to its extended position (shown in the figure), stapling and cutting a hole in the captured tissue plication and pushing the hooked end of pin 244 into the webbing in the anchor cap, to engage the pin with the anchor as shown.

The drive piston is now retracted to pull the anchor from the anvil cavity and into placement in the plication hole, with the anchor cap and plate on opposite sides of the hole. As a final step, pin 244 is disengaged from the anchor, e.g., by heating the hooked end of the pin as it is pulled away from the anchor, or gently rotating the pin as it is retracted.

FIG. 15 illustrates an embodiment of the invention in which the anchor is assembled from its separate cap and plate parts by a rivet connection that occurs during tissue stapling. In this embodiment, the anchor cap is carried in the staple member and the anchor plate in the anvil member, e.g., on a cutter board surface within the anvil. When the two members come together, activation of the staple pusher is also effective to move the anchor cap into and through the tissue plication, to engage the anchor plate, and form an anchor in the stapled plication.

Arm Assemblies

Following is a discussion of the features of the arm assemblies 32, FIG. 5 shows the arm assemblies 32 separated from the other elements of the stapler head. In general, each arm assembly has a first arm section 100 pivotally coupled to the staple housing and a second arm section 102 pivotally coupled between the first arm section and the anvil housing. While not present in the illustrated embodiment, additional arm sections may be positioned between the first and second arm sections.

That is, each arm assembly includes a proximal arm 100 and a distal arm 102 joined to one another to form a hinge 104. Each of the proximal arms 100 has a longitudinal cutout 108 and an arm spreader 113 pivotally mounted within the cutout 108. The distal end of each arm spreader 113 includes a bore 112. Pin 84 is positioned within the bore 112. As disclosed in connection with FIG. 10, this pin 84 extends through the disk 68 and has ends that ride within the slots 64 (FIG. 6) on the lower and upper sections of the stapler housing. Longitudinal movement of the disk 68 within the stapler housing will thus advance the pins 84 within their corresponding slots 64, causing the arm spreaders 113 to pivot relative to the pins 84 and to thus drive the arm assemblies 32 outwardly. Additional specifics concerning movement of the arm assemblies 32 is set forth in the section entitled Stapler Head Operation.

Distal arms 102 of the arm assemblies include pins 36 which, as discussed, are pivotally mounted to the anvil housing 30 (FIG. 4). A pair of drive links 114 are provided, each of which has a first end pivotally attached to a corresponding one the distal arms 102 and a second end pivotally coupled to a common pin 116. In the assembled stapler head, pin 116 is positioned in the bores 93 of the upper and lower plates 95a, 95b of the anvil support (see plates 95a, b in FIG. 12). As detailed in the Stapler Head Operation section below, when the arm spreaders 113 drive the arm assemblies 32 outwardly, drive links 114 act on the pin 116 to push the anvil support in a proximal direction, causing the anvil to advance proximally towards the staple cartridge.

Stapler Head Operation

The following discussion centers on the manner in which the arm assemblies function to expand the vacuum chamber and to compress tissue that has been drawn into the chamber using suction. As an initial step preceding chamber expansion, the stapler head is positioned with the opening 26 in the membrane 24 in contact with tissue at the location at which plication creation is desired. Vacuum source 20 (FIG. 3) is activated to apply vacuum to the inside of the vacuum chamber defined by the membrane. Tissue in contact with the opening 26 (FIG. 4B) will be drawn into the vacuum chamber between the staple housing 28 and the anvil housing 30. After the tissue is drawn in, the stapler profile is changed, expanding the volume of the chamber within the membrane.

The streamlined position of the stapler head 28 prior to expansion is shown in FIG. 5. In particular, the hinged arm assemblies 32 and membrane raisers 37 are in generally straight orientations. The proximal arms 100 serve as the drive arms for chamber expansion and tissue compression. Motion of these arms is initiated when water under pressure is forced into the hydraulic circuit of the staple housing. Referring to FIG. 7, fluid pressure to piston 106 advances disk 68, which in turn pushes the staple cartridge 78 toward the anvil 96 as shown in FIGS. 19-21, causing the staple cartridge 78 to extend further from the staple housing 28.

Both the disk 68 and the arm spreaders 113 are coupled to the pins 84. For this reason, the longitudinal movement of the disk 68 within the stapler housing 28 will carry the pins 84 distally within their corresponding slots 64. The arm spreaders 113 will consequently pivot relative to the pins 84, driving the proximal arms 100 outwardly. Outward movement of proximal arms 100 at hinge 104 causes the distal arms 102 to also pivot outwardly at hinge 104, forming an angle between the proximal and distal arms 100, 102. Naturally, formation of the angle between the arms 100, 102 shortens the effective length between the remote ends of the arms, causing the distal pins 36 of the distal arms 102 to carry the anvil housing 30 towards the staple cartridge. The pivoting movement of the distal arms 102 further causes drive links 114 to act on pin 116 to push the anvil support in a proximal direction. This moves the anvil support relative to the anvil housing in a proximal direction at the same time the anvil housing is also moving proximally.

In essence, one motion, that of the hydraulically driven compression piston, creates at least three motions: the staple cartridge 78 moving relative to the staple housing in a direction towards the anvil 96, the anvil housing 30 moving toward the staple housing 28 and the anvil 96 itself moving relative to the anvil housing 30 in a direction towards the cartridge. This compound motion of the anvil toward the staple cartridge enables a small displacement of the compression piston to quickly compress tissue in the grip of stapler. The multiplication of motion also enhances force transmission between the two housings by keeping the angle at hinge 104, between the proximal (driven) arm and the distal (drive) arm, as large as possible.

The relative motion of the two housings 28, 30 toward each other also drives upward links 38, 40 and their interconnecting spring wires 46 on the top of the stapler head 14. Together, the links and spring wires raise the top of the membrane, creating more volume to accommodate expansion of the tissue during compression.

Compression of the tissue is halted when the pins 84 traveling in slots 64 in the staple housing 28 reach the limit of travel. Thus, the slots and associated components are dimensioned to set the desired separation distance between the tissue contact surfaces on the stapler side and the anvil side of the stapler head. Exemplary separation distances for use in stomach wall plications might include approximately 0.06-0.07 inches (e.g. for use with staples having legs of 5.5 mm length) or 0.109 inches for 6.5 mm leg length staples. Application of additional pressure into the hydraulic circuit will not compress the tissue any further.

Moreover, because of the piston arrangement, the stapling function is effectively locked out until tissue compression is complete. With this arrangement, fluid introduced via the fluid port 50b (FIG. 6A) into the staple fluid channel 122 prior to completion of tissue compression will leak until the two o-rings 112 of the compression piston 106 are straddling the inlet 114. This design prevents premature staple firing.

At the fully compressed position, the arm spreaders 113 are nearly perpendicular to the longitudinal centerline of the stapler head. Once tissue is compressed between cartridge 78 and anvil 96, the tissue is ready for stapling.

Stapling is initiated by introducing hydraulic fluid to the device. The staple piston advances, pushing cutting element 86 towards the anvil 96. Because the staple pusher 76 is mounted to the cutter 86, this action carries the staple pusher 76 through the cartridge 78 where it simultaneously pushes all staples through the tissue. Staple piston travel is limited by internal stops, and is preset to yield optimal staple formation.

During compression, as the angle at the hinge 104 of arm assemblies 32 reaches its minimum, the force required to resist separation of the staple and anvil housings increases. These forces increase further when the forces of staple crushing are exerted on the anvil by the staple piston. To compensate, the arm spreaders 113 serve as displacement struts to channel at least a portion of these forces into the disk 68. These forces, if not reacted by the pusher disk, would pull in the arms 100, 102 and potentially release the compression on the tissue, causing incomplete staple formation or tissue cutting. In this way, a truss-like structure is created for force displacement.

When staples have been formed, staple pressure is released and a spring (not shown) returns the staple pusher 72 to its base position. Releasing fluid pressure will allow the deflected spring wires 46 on membrane raiser 37 to return the staple head to its minimum profile configuration and release the plication from the stapler. Once outside the patient, the used staple cartridge can be ejected and a new one installed.

Anchor Mechanism and Placement

In operation, and with reference to FIGS. 16A-16F, as the two stapler members are brought together, a tissue fold is captured between the outer face of the staple cartridge and the confronting face of the anvil. As an initial step (FIG. 16A), endoscopic guide tube 23 is advanced into the stomach via the mouth and esophagus. The endoscope 22 is inserted into the endoscope channel in the stapler handle (not shown) and advanced down the lumen of the stapler handle. The stapler/endoscope are simultaneously passed through the endoscopic guide tube towards the stomach. Once the stapler and endoscope reach the gastroesophageal junction region of the stomach, the position of the stapler is maintained while the endoscope is advance further into the stomach.

The stapler head 14 is advanced to the desired depth and location in the stomach. Using the articulation controls on the stapler handle, the angular orientation of the stapler head is adjusted to allow positioning of the stapler head 12 at the pre-identified target tissue as shown in FIG. 31A. The opening 26 in the membrane 24 is positioned against the target tissue. The endoscope 22 is placed in a retroflexed position as shown.

The vacuum source 20 is coupled to the vacuum port on the handle external to the body, and vacuum pressure is applied to draw tissue 17 through the opening 26 and into the vacuum chamber defined by membrane 24 as shown in FIGS. 31B and 32A. Acquisition of the target tissue will be readily identified endscopically through the wall of transparent membrane 24 on the stapler head.

The fluid source (is shown) is coupled to the handle. Once it has been visually confirmed that a sufficient amount of tissue has been acquired, fluid is introduced to cause compression of the tissue and expansion of the arm assemblies 32 and membrane raiser 37 as shown in FIGS. 16C and 16D. As can been seen, the expansion of the arm assemblies and the membrane allows a large volume of tissue to be acquired into the vacuum chamber and displaced further into the chamber during tissue compression. As noted earlier, the drawing in of tissue into the expanded chamber during operation, well “above” the staple holder and anvil provides a relatively large margin of tissue around the stapled portion of the tissue, reducing the risk of tissue tearing or tissue-fold weakness near the stapled portion of the tissue. The captured tissue fold is indicated at 17a.

Once the tissue has been compressed, additional hydraulic fluid is introduced to cause stapling and cutting of the tissue as shown in FIG. 16D, forming a plication P, indicated at 17b in FIG. 17. At the same time, the engagement assembly moves distally to engage the plug in the anchor assembly. The compression and stapling hydraulic sources are then deactivated to release fluid pressure within the hydraulic circuit, also causing the anchor plug to be released from the anvil member, and then pulled through the hole formed in the stapled plication. With the hydraulic pressure relieved, the spring wires of the membrane raiser 37 help to restore the stapler head 14 to its original streamlined configuration, allowing the stapler head to be withdrawn from the tissue as shown in FIG. 161E, with the plug still attached to the engagement pin of the device, and the anchor tether to the plug on the other side of the tissue fold. Using the endoscope for visualization, the stapling device is now moved within the stomach to pull the anchor through the tissue plication hole, as shown in FIG. 16F, Following this step, the tether is cut, leaving a stapled tissue plication with an anchor placed therein.

In a preferred plication configuration shown in FIG. 17 the staples 158 are arranged in two concentric rings of five staples, with the staple reinforcement device 83 retained by the staples and distributing forces around the staple pattern as shown. The plication P includes a hole H formed by the cutting element, through which various implants or anchors for various implants can be placed.

If multiple plications are needed, the stapler 12 is briefly withdrawn from the endoscopic guide tube and the staple cartridge is replaced, and a new anchor assembly is loaded onto the device.

The system may be packaged with instructions for use instructing the user to use the various disclosed features to perform a stapling procedure using methods disclosed herein.

The disclosed systems provide convenient embodiments for carrying out the disclosed compression, stapling and plication tagging functions. However, there are many other widely varying instruments or systems may alternatively be used within the scope of the present invention, Moreover, features of the disclosed embodiments may be combined with one another and with other features in varying ways to produce additional embodiments. Thus, the embodiments described herein should be treated as representative examples of systems useful for forming endoscopic tissue plications, and should not be used to limit the scope of the claimed invention.

Any and all patents, patent applications and printed publications referred to above, including those relied upon for purposes of priority, are incorporated herein by reference.

It is claimed:

1. In a stapling device designed to capture a tissue fold between first and second members in the device and to staple and cut the fold to form a stapled tissue plication with a hole therein, an improvement for placing in the stapled tissue plication, an anchor effective to attach an implant device to the tissue fold, comprising
an engagement assembly in the first member movable from a retracted position in the first member to an extended position in the second member, and
releasably attached to second member, an anchor assembly that includes the anchor,
wherein (i) movement of the engagement assembly from its retracted to its extended position, through a tissue fold captured in the device between the first and second device members, is operable to engage the anchor assembly, and (ii) movement of the engagement assembly, with the anchor assembly engaged therewith, back toward its retracted position, is effective to pull at least a portion of the assembly through the hole in the stapled tissue plication formed by the device.

2. The improvement of claim 1, wherein the first member includes a housing and a drive piston movable within the housing from a retracted position to an extended position and a tissue cutter, the engagement assembly includes an engagement pin, the cutter and pin are attached to the drive piston for movement therewith, and movement of the drive piston from its retracted to its extended position is operable to (i) drive the pin through a tissue fold captured in the device, (ii) form a hole in the tissue fold, and (iii) engage the pin with the anchor assembly.

3. The improvement of claim 2, wherein the first member includes a staple holder having a first tissue-contact surface adapted to contact one face of a captured tissue fold and a staple pusher attached to the drive piston for movement therewith within the first-member housing, and movement of the drive piston from its retracted to its extended position is operable to (i) staple the tissue fold captured in the device, (ii) drive the pin through a fold, (iii) form a hole in the tissue fold, and (iv) engage the pin with the anchor assembly.

4. The improvement of claim 3, wherein the second member includes a housing, an anvil attached to the housing and having a second tissue-contact surface adapted to contact the other face of a captured tissue fold, and a cutter board mounted adjacent the anvil, against which the cutter is pressed when the cutter is moved to its second position, to cut a hole in a tissue fold captured between the tissue-contact surfaces of the staple holder and anvil.

5. The improvement of claim 4, wherein the anchor assembly includes (i) a pin-engaging plug mounted releasably on the second-member housing adjacent the cutter board, (ii) an anchor and (iii) a tether that attaches the plug to the anchor, the cutter board includes slot in which the tether is received, below the surface of the board, and movement of the engagement pin from its extended toward its retracted position, after engagement with the plug, is effective to (i) release the plug from the second member, (iii) pull the plug through a hole formed by the device in the stapled tissue plication, and (iii) remove the tether from its slot in the cutter board.

6. The improvement of claim 5, wherein the engagement pin and plug are designed to allow the pin to penetrate a portion of the plug, as the pin is moved toward its extended position, but prevent disengagement of the pin with the plug when the pin is moved toward its retracted position.

7. The improvement of claim 5, wherein the device includes a membrane connecting the two members, defining a tissue-capture chamber therebetween, and an opening in the membrane through which tissue can be drawn into the chamber, under the influence of a vacuum applied to the chamber, the anchor in the anchor assembly is held releasably on the device outside the chamber, the tether connecting the plug to the anchor extends through the opening in the membrane, and movement of the engagement pin from its extended to retracted position, after engagement with the plug, is effective to (i) release the plug from the second member, (iii) pull the plug through a hole formed in the stapled tissue plication, (iii) remove the tether from its slot in the cutter board, and (iv) release the anchor from the device, wherein the device can be manipulated to pull the anchor through the hole in the stapled tissue plication after the stapled tissue plication has been released from the device.

8. The improvement of claim 7, wherein the anchor includes a front, compressible cap joined to a back plate, and manipulating the device to pull the anchor through a hole in a staple tissue plication is effective to pull the cap through the hole, to engage one side of the fold, with the plate supported against the other side of the fold.

9. The improvement of claim 7, wherein the device further includes means for severing the anchor-assembly tether, once the anchor is pulled through the hole in the stapled tissue plication.

10. The improvement of claim 7, wherein the tether includes is designed to separate upon application of a predetermined pulling force applied to the tether.

11. The improvement of claim 7, wherein the two members in the device are relatively movable toward one another to capture a tissue fold therebetween, and relatively movable away from one another to release a stapled tissue plication from the device, prior to manipulating the device to pull the anchor through the hole formed in the stapled tissue plication.

12. The improvement of claim 4, wherein the anchor assembly includes a front, compressible cap joined to a flexible back plate, the anchor is carried releasably within the second-member housing, movement of the engagement pin from its retracted to extended position is effective to engage the anchor, and movement of the engagement pin from its extended toward its retracted position is effective to (i) release the anchor from the second-member housing, and (ii) pull the anchor cap through the hole in the stapled tissue placation formed by the device, to engage one side of the fold, with the anchor plate supported against the other side of the fold.

13. The improvement of claim 12, wherein the second-member housing includes a central cavity having an opening surrounded by the cutter board and anvil, and the anchor assembly is releasably contained within said cavity.

14. The improvement of claim 13, wherein said engagement pin includes means for releasing the anchor after the anchor's compressible cap has been pulled through the hole in the stapled tissue plication formed by the device.

15. A method for forming a stapled tissue plication having placed therein, an anchor effective to attach an implant device to the tissue fold, comprising
(a) capturing a tissue fold between two tissue-contact members,
(b) stapling the captured tissue so as to form a pattern of staples about an anchor zone,
(c) extending an engagement assembly through the anchor zone, before, during, or after said stapling step, to engage an anchor assembly,
(d) before, during or after said extending step, forming a hole in said anchor zone,
(e) retracting the engagement assembly and engaged anchor assembly to pull at least a portion of the anchor assembly through the hole at the tissue anchor zone, and
(f) releasing the stapled tissue from the tissue-contact members.

16. The method of claim 15, wherein the engagement assembly includes an engagement pin, the anchor assembly includes a pin-engaging plug, an anchor having a front, compressible cap and a back plate, and a tether connecting the plug to the anchor, step (c) is effective to engage the pin with the plug, and step (e) is effective to pull the plug through the hole formed in the tissue in step (d).

17. The method of claim 16, which further includes, following step (f), manipulating the engagement pin to pull the anchor cap through the hole formed at the anchor zone in the stapled tissue plication, to engage one side of the fold, with the anchor plate supported against the other side of the fold, and cutting the tether to release the stapled tissue plication with placed anchor from the pin and engaged plug.

18. The method of claim 17, wherein the engagement assembly includes an engagement pin, the anchor assembly includes a front, compressible cap and a back plate, and a tether connecting the plug to the plate, step (c) is effective to engage the pin with the anchor, and step (e) is effective to pull the plug through the hole formed in the tissue in step (d), to engage one side of the fold, with the anchor plate supported against the other side of the fold, and which further includes disengaging the engagement pin from the anchor prior to releasing step (f).
